(19) United States
(12) Patent Application Publication
(10) Pub. No.: US 2008/0069790 A1
(43) Pub. Date: Mar. 20, 2008

(54) Pre-Shave Preparation With Enhanced Lubricity Using Carboxy Silicone Polymer

(76) Inventors: Teresa Petraia, Henderson, NV (US); Eric Spengler, Ridgefield, CT (US)

Correspondence Address: ST. ONGE STEWARD JOHNSTON & REENS, LLC, 986 BEDFORD STREET, STAMFORD, CT, 06905-5619, US

(21) Appl. No.: 11/856,976

(22) Filed: Sep. 18, 2007

Related U.S. Application Data
(60) Provisional application No. 60/845,957, filed on Sep. 20, 2006.

(51) Int. Cl. A61K 8/58; A61Q 9/02

(52) U.S. Cl. 424/70.12

(57) ABSTRACT

Pre-shave compositions to lubricate skin during electric shaving that comprise at least one carboxy silicone polymer and a volatile component to serve as a carrier to the polymer. The pre-shave composition lubricates the skin to reduce skin friction between the electric razor and the skin surface, provides a closer shave, and provides improved feeling to the skin during and after shaving.

### CROSS-REFERENCE TO RELATED APPLICATIONS

The present application claims the benefit under 35 U.S.C. §119(e) of the U.S. Provisional Patent Application Ser. No. 60/845,957, filed on Sep. 20, 2006, the content of which is incorporated herein by reference.

### FIELD OF THE INVENTION

The present invention relates to compositions that prepare the skin for shaving, in particular to pre-shave compositions utilized with an electric razor that reduce friction between the skin's surface and the electric razor, optimize shaving performance and improve the feeling of the skin.

### BACKGROUND OF THE INVENTION

When consumers use a pre-shave product in conjunction with an electric razor, they can sense the friction between the skin and the razor head(s). Friction can lead to uncomfortable skin irritation. But more importantly, friction between the razor head and skin will distort skin in front of the razor causing it to “bunch up” or create “a wave.” Thus changing the angle of the razor head to the beard hair being cut and greatly reducing the efficiency of the shaving process—possibly leading to a poor shave.

Compositions applied to the skin prior to shaving known in the art generally seek to prepare the skin to receive a razor, providing lubrication and reducing friction to prevent irritation or cutting of the skin. This is achieved in different ways by the two most popular forms of pre-electric shave preparations: the lotion based on an alcoholic solution and the talc stick.

In formulating a pre-electric shave lotion the following attributes are considered desirable: 1) adequate astringency to stiffen the beard and possibly to stimulate the hair follicle muscles; and 2) provision of a coating on the skin on which the razor will glide, thereby preventing irritation of the skin and providing lubrication for the cutting edge of the electric razor. To achieve this, some alcoholic preparations can contain 5% to 20% of fatty acid esters such as Isopropyl Myristate to aid in the glide of the razor. Powders, such as talc, nylon, polystyrene, polyethylene, and polyester have been employed to increase lubricity. European Patent number 0 385 312 A2 speaks to a pre-shave preparation for an electric shaver comprising of a globular powder. The globular powder refers to nylon, polystyrene, polyethylene, etc. The globular powder is dispersed in an alcohol vehicle and is shaken prior to use.

What is needed is a pre-shave preparation with outstanding lubricity to provide a closer shave and reduce skin friction. It is a further object of the invention for the preparation to (I) lubricate the skin for reduced skin friction between the electric razor and the skin surface, (II) provide a closer shave, and (III) provide improved skin feel during and after shaving.

### SUMMARY OF THE INVENTION

These objectives are achieved by a pre-shave composition containing a carboxy silicone. In particular the composition can contain (1) a carboxy silicone polymer such as Cetyl Triethylmonium Dimethicone PEG-8 Succinate, and (2) a volatile component such as alcohol to serve as a carrier to the polymer.

It is another aspect of the invention for the composition to contain a fatty acid ester with emollient properties.

It is a further aspect of the invention for the composition to comprise at least one adjunct ingredient selected from the group consisting of skin conditioners, emollients, humectants, color, fragrance, antioxidants, chelators, natural extracts, vitamins, UV light absorbers, solvents and combinations thereof.

Other objects of the invention and its particular features and advantages will become more apparent from consideration of the accompanying detailed description.

### DETAILED DESCRIPTION OF THE INVENTION

The present invention is directed to a pre-shave preparation that utilizes water soluble silicone polymers, in particular polyethylene glycol derivatives of dimethicones—for example PEG 8 Dimethicone. These compounds are very water and alcohol soluble. These silicones can impart lubricity that are beneficial for use with pre-shave preparations. Polyethylene glycol derivatives of dimethicones are effective in such an application because they do not significantly reduce skin friction properties on skin relative to existing art.

A new class of water and alcohol soluble silicone derivatives are carboxy silicone polymers as described in U.S. Pat. No. 6,867,317 B1. This ingredient class is not used in pre-electric shave products and has not been known to have special lubricating properties beyond ethoxylated silicones nor are they known to provide a closer shave relative to existing art. However testing has surprisingly shown this class to provide both better skin friction reducing properties that also yields a closer shave relative to existing art.

One example of a carboxy silicone is cetyl triethylmonium dimethicone PEG-8 succinate. Cetyl triethylmonium dimethicone PEG-8 succinate is a siloxane polymer formed by the reaction of cetyl triethylamine with PEG-8 dimethicone (q.v.) and succinic acid (q.v.) having the general formula of:

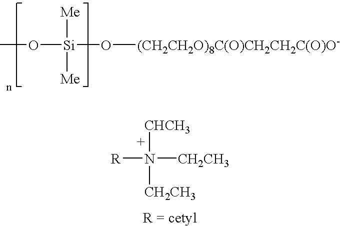

Testing has demonstrated that this class of compounds significantly reduces skin friction to a greater extent than its parent backbone—PEG-8 Dimethicone while maintaining very clear water/alcohol solutions. Preferred concentrations of a carboxy silicone polymer are 0.05% to 20%.

In addition to cetyl triethylmonium dimethicone PEG-8 succinate, compounds of the following general structure are useful as reducers of friction in pre-shave preparations.

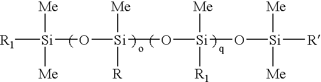

Wherein;
Me is methyl;

R and R′ are CH₃ or —(CH₂)₃—O-(EO)ₐ—(PO)_{b}-(EO)_{c}—O—R″—C(O)—O— or

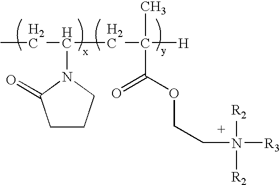

With the proviso that both R and R′ are not CH₃;

R₂ and R₃ are independently either methyl or ethyl;

x is an integer from 1 to 100;

y is an integer from 1 to 100;

R″ is selected from a group consisting of —CH₂CH₂—; —CH═CH—; —CH₂—C(R⁷)—H₂;

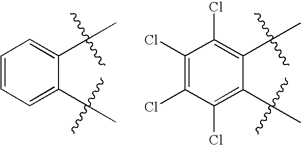

R₁ is selected from the group consisting of lower alkyl CH₃(CH₂)ₙ— and phenyl;

n is an integer from 0 to 8;

a, b, and c are integers independently ranging from 0 to 20;

EO is an ethylene oxide residue —(CH₂CH₂—O)—;

PO is a propylene oxide residue —(CH₂CH₂CH₂—O)—;

o is an integer from 1 to 100;

q is an integer from 0 to 500;

R⁷ is alkyl having from 1 to 20 carbons.

A second important component for a liquid pre-shave preparation is a volatile carrier such as ethanol. Other acceptable volatile carriers are described in the European Patent Application #0385312 A2. Ethanol or ethyl alcohol is a volatile liquid used in cosmetic preparations. It is derived from ethylene by direct catalytic hydration or with ethyl sulfate as an intermediate. For purposes of testing, as discussed below, Specially Denatured Alcohol (SDA) 40B was chosen, which contains Bitrex and tert-butyl alcohol.

The content of the above volatile carrier is at least 50% by weight, preferably at least 70% by weight. When the content is less than 50% by weight, the drying characteristics of the pre-shave preparation after application is poor. This can be detrimental to the convenience and speediness in-use of the electric shaver: the higher the volatile component the faster the evaporation of moisture on the skin. In addition, the alcohol partially dehydrates some of the moisture normally present in the beard. The partial dehydration has an important effect in stiffening the beard to permit easier cutting.

An optional component of this invention is a Fatty Acid Ester. These compounds are commonly used in commercially available pre-shave preparations. The most common ester is Isopropyl Myristate with a chemical formula:

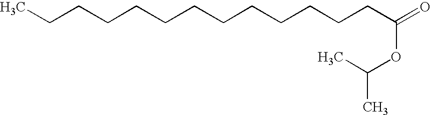

An ester is formed by the condensation reaction between an acid and an alcohol. One or both of these components needs to be fatty in nature to produce an ester with emollient properties. The chain length of either the acid portion or the alcohol portion of an ester can be varied. For example, keeping the alcohol portion of an ester the same (as in Isopropyl Myristate) and increasing the chain length of the acid raises the melting point and makes the material more hydrophobic. As the size of the chain length increases, the ester loses its fluidity and becomes a solid at room temperature. Other attributes which may affect the esters function are molecular weight, chain branching, polarity and saturation versus unsaturation. A liquid emollient ester is preferred which is soluble in a water/alcohol solution and does not interfere with skin friction. Other such esters are C12-15 Alkyl Benzoate or PPG-3 Benzyl Ether Myristate.

Other ingredients suitable for use in a pre-shave hydro-alcoholic preparation include, without limitation, skin conditioners, emollients (including esters and silicones), humectants, color, fragrance, antioxidants, chelators, natural extracts, vitamins, UV light absorbers, solvents and combinations thereof.

Friction Testing

When consumers use a pre-shave product in conjunction with an electric razor, they can sense the friction between the skin and the razor head(s). Friction can lead to uncomfortable skin irritation. But more importantly, friction between the razor head and skin will distort skin in front of the razor causing it to “bunch up” or create “a wave.” Thus changing the angle of the razor head to the beard hair being cut and greatly reducing the efficiency of the shaving process—possibly leading to a poor shave.

Friction testing was performed on pre-shave compositions containing a carboxy silicone polymer. This testing utilized a skin friction meter that presses a probe against a synthetic skin surface and measures the force required to either push or pull the probe on the surface. The skin friction meter measures the coefficient of friction for each tested sample.

Methodology

An instrument known as the Skin Friction Meter designed by Measurement Technologies (Cincinnati, Ohio) is available through Aca-Derm, Inc of Menlo Park, Calif. The instrument is a rotary disk instrument and consists of three main parts: a probe unit, a stationary shell and a rotary disc transducer. The probe unit consists of a small DC motor with a Teflon disk type probe attached. It is mounted inside the stationary shell between two ball bearings and is connected to the shell by a coil spring. The rotary probe transducer is mounted on the end of the stationary shell and is joined to the end of the probe unit by a soft coupling and monitors the position of the probe unit. Since the unit has a hard probe, it may be used to measure most skin friction phenomena as is.

The instrument is designed to be hand held; and for maximum flexibility, is connected to its electronic controller by a six foot cable. For hand held use, the following design innovations are used to control application pressure. The instrument rests on the measurement area on a Lexan® plastic base plate which has a hole in the center. When resting on the skin surface, the application force causes the skin and underlying tissues to protrude through the hole. Application pressure on the probe itself is controlled by its position relative to the hole in the plastic base plate. Therefore, since the hole in the base plate is constant and the probe position is constant, when the measurement head rests on the measurement site with only its own weight, probe contact pressure will remain constant. In order to keep measurements within the linear range of the transducer, probe application pressure may be either increased or decreased by changing the position of the probe relative to the base plate.

The probe is easily adjusted to accommodate measurements which are either too low or too high. Motor speed is 69.4 rpm maximum and may be manually controlled from the front panel of the control box. The analog output of the instrument in its most linear range is a 6 volt d.c. range from −3.0 to 3.0 volts. Torque or force applied to the probe is measured and displayed as friction meter units. The higher the unit the greater the “friction value”.

Friction measurements were taken using a synthetically produced “skin” called VITRO-SKIN as supplied by IMS Inc. (Orange, Conn.). The “skin” was cut into 2×2 cm squares and placed in a hydration chamber according to IMS directions. Baseline readings were taken without application of the pre-shave preparation. A determined amount of the pre-shave preparation (10 μl) was applied to the site using a micropipette and allowed to dry for 15 seconds. The probe was then placed on the site and measurements were taken after 30 and 90 sec. The latter value corresponds to an extended shaving process.

The following table identifies pre-shave composition samples that underwent friction testing:

TABLE 1
Pre-Shave Composition Samples:

1. Isopropyl Myristate/Alcohol
2. Cetyl Triethylmonium Dimethicone PEG-8 Succinate/Alcohol solution
3. Cetyl Triethylmonium Dimethicone PEG-8 Succinate/Alcohol solution
4. Cetyl Triethylmonium Dimethicone PEG-8 Succinate/Alcohol solution
5. Cetyl Triethylmonium Dimethicone PEG-8 Succinate/Isopropyl
Myristate/Alcohol
6. PEG-8 Dimethicone/Isopropyl Myristate/Alcohol

Sample 1 represents a currently marketed pre-shave product.

The following table lists the composition of each pre-shave sample:

TABLE 2

| % Composition | Sample 1 | Sample 2 | Sample 3 | Sample 4 | Sample 5 | Sample 6 |
|---|---|---|---|---|---|---|
| SD Alcohol | 83.0 | 99.5 | 95.0 | 90.0 | 85.0 | 85.0 |
| Isopropyl | 17.0 | | | | 14.0 | 14.0 |
| Myristate | | | | | | |
| Cetyl | | 0.5 | 5.0 | 10.0 | 1.0 | |
| Triethylmonium | | | | | | |
| Dimethicone | | | | | | |
| PEG-8 | | | | | | |
| Succinate | | | | | | |
| PEG-8 | | | | | | 1.0 |
| Dimethicone | | | | | | |

For each sample, a baseline coefficient of friction measurement was taken on the synthetic skin surface without a pre-shave preparation. Then the sample was applied to the surface and coefficient of friction measurements were taken of the surface at 30 seconds and 90 seconds after the sample was applied. Each sample was tested three times for each period. The following table lists the average coefficient of friction measurement for the baseline, each period and the percent change in friction due to the application of each pre-shave preparation relative to the baseline:

TABLE 3

| | | 30 | | 90 | |
|---|---|---|---|---|---|
| Sample | Baseline | seconds | % Δ | seconds | % Δ |
| 1 | 3.6 | 1.9 | 47.2 | 1.8 | 50.0 |
| 2 | 8.7 | 2.1 | 76.0 | 1.6 | 81.6 |
| 3 | 6.4 | 0.9 | 86.0 | 0.6 | 90.6 |
| 4 | 8.4 | 0.7 | 91.7 | 0.7 | 91.7 |
| 5 | 9.0 | 1.4 | 84.4 | 1.2 | 86.6 |
| 6 | 4.1 | 3.4 | 17.0 | 3.3 | 19.5 |

Table 3 shows that compositions containing a carboxy silicone significantly reduce skin friction values. The reduction in friction is greater than compositions containing a fatty acid ester or a water/alcohol soluble silicone—a polyethylene glycol derivative of dimethicone (PEG 8 Dimethicone).

Evaluation of Shave Performance

Samples 1 and 5 were also utilized to test actual performance in improving shaving and compare the results with shaving without a pre-shave preparation. 21 males, ages 18-65, were utilized to test the two samples. The study design consisted of a split-face (treated versus non-treated) cross-over design, with a right/left randomization of treated and untreated. In this manner each subject generated his own internal no treatment control site. After shaving, each surface was evaluated for ease of shaving and overall skin smoothness. 24 hours later hair length measurements were taken to assess the closeness of the shave. The following table shows the percent change in beard hair length for a skin surface that utilized either pre-shave preparation versus a skin surface that did not utilize a pre-shave preparation:

TABLE 4

| | Sample 1. | Sample 5. |
|---|---|---|
| % Closer Shave | 6.33 | 15.45 |
| (reduction in beard hair length | | |
| compared to no pre-shave control) | | |
| Std Dev | 59.82 | 46.07 |

The following table shows the subjective evaluation of ease of shaving and smoother skin for each skin surface:

TABLE 5

| | | % of panel who | | % of panel who |
|---|---|---|---|---|
| | % of panel who | selected the no | % of panel who | selected the no |
| | selected Sample 1 | treatment side as | selected Sample 5 | treatment side as |
| | as better than no | better than the | as better than no | better than the test |
| | treatment | test product | treatment | product |
| Smoother | 57% | 19% | 62% | 14% |
| Easier to | 62% | 10% | 67% | 10% |
| Shave | | | | |

The preceding results demonstrate that carboxy silicones provide significant reduction of friction between the shaver head and skin; and that preparations using carboxy silicones provide a significantly closer shave when compared to a no pre-shave treatment.

Although the invention has been described with reference to pre-shave preparations containing particular elements or compositions and particular relative amounts, these are not intended to exhaust all possible arrangements or features, and indeed many other modifications and variations will be ascertainable to those of skill in the art.

What is claimed is:

1. A composition to lubricate the skin during electric shaving, comprising:
at least one carboxy silicone polymer;
and a volatile component to serve as a carrier to the polymer.

2. The composition of claim 1, wherein the carboxy silicone polymer comprises about 0.05% to about 20% by weight of the composition.

3. The composition of claim 1, wherein the carboxy silicone polymer comprises about 0.1% to about 10% by weight of the composition.

4. The composition of claim 1, wherein the carboxy silicone polymer comprises about 0.5% to about 5% by weight of the composition.

5. The composition of claim 1, wherein the volatile component comprises about 50% to 99.95% by weight of the composition.

6. The composition of claim 1, wherein the volatile component comprises about 60% to 95% by weight of the composition.

7. The composition of claim 1, wherein the carboxy silicone polymer is Cetyl Triethylmonium Dimethicone PEG-8 Succinate.

8. The composition of claim 1, wherein the volatile component is an alcohol.

9. The composition of claim 1, further comprising at least one adjunct ingredient selected from the group consisting of skin conditioners, emollients, humectants, color, fragrance, antioxidants, chelators, natural extracts, vitamins, UV light absorbers, solvents and combinations thereof.

10. The composition of claim 1, wherein the composition provides a closer shave and an improved feel to the skin after shaving is completed.

11. A composition to lubricate the skin during electric shaving, comprising:
at least one carboxy silicone polymer;
a volatile component to serve as a carrier to the polymer; and
a fatty acid ester with emollient properties.

12. The composition of claim 11, wherein the fatty acid ester comprises about 1% to 50% by weight of the composition.

13. The composition of claim 11, wherein the fatty acid ester comprises about 10% to 30% by weight of the composition.

14. The composition of claim 11, wherein the fatty acid ester is Isopropyl Myristate.

15. A composition to reduce skin friction between the electric razor and the skin surface, comprising:
a carboxy silicone polymer;
and a volatile carrier.

16. The composition of claim 15, wherein the carboxy silicone polymer comprises about 0.05% to about 20% by weight of the composition.

17. The composition of claim 15, wherein the carboxy silicone polymer comprises about 0.1% to about 10% by weight of the composition.

18. The composition of claim 15, wherein the carboxy silicone polymer comprises about 0.5% to about 5% by weight of the composition.

19. The composition of claim 15, wherein the volatile component comprises about 50% to about 99.95% by weight of the composition.

20. The composition of claim 15, wherein the volatile component comprises about 60% to about 95% by weight of the composition.

21. The composition of claim 15, wherein the composition provides an improved skin feel to the skin after shaving is completed.

22. The composition of claim 15, wherein the composition provides a closer shave and an improved feel to the skin after shaving is completed.

23. A composition to reduce skin friction between the electric razor and the skin surface, comprising:
a carboxy silicone polymer;
a volatile carrier; and
a fatty acid ester.

24. The composition of claim 23, wherein the carboxy silicone polymer comprises about 0.05% to about 20% by weight of the composition.

25. The composition of claim 23, wherein the carboxy silicone polymer comprises about 0.1% to about 10% by weight of the composition.

26. The composition of claim 23, wherein the carboxy silicone polymer comprises about 0.5% to about 5% by weight of the composition.

27. The composition of claim 23, wherein the alcohol comprises about 50% to about 99.95% by weight of the composition.

28. The composition of claim 23, wherein the alcohol comprises about 60% to about 95% by weight of the composition.

29. The composition of claim 23, wherein the fatty acid ester comprises about 5% to about 40% by weight of the composition.

30. The composition of claim 23, wherein the fatty acid ester comprises about 10% to about 30% by weight of the composition.

31. The composition of claim 23, wherein the composition provides a closer shave and an improved feel to the skin after shaving is completed.

32. A composition to provide a closer shave between the razor and the skin surface, comprising:
about 0.1% to about 10% of carboxy silicone polymer;
at least about 60% by weight alcohol as a carrier to the carboxy silicone polymer; and
about 10% to about 30% of a fatty acid ester.

33. The composition of claim 32, wherein the composition provides a closer shave and an improved feel to the skin after shaving is completed.
